(19) United States
(12) Patent Application Publication
(10) Pub. No.: US 2005/0026855 A1
(43) Pub. Date: Feb. 3, 2005

(54) Therapeuticum and prophylacticum for malignant diseases using phenotype suppression

(76) Inventors: Viktor Balazs, Odenthal (DE); Margit Frohlich, Odenthal (DE)

Correspondence Address: DAVID A. GUERRA, 933 OLANDER WAY SOUTH, SUITE 3, SOUTH PASADENA, FL, 33707, US

(21) Appl. No.: 10/498,795

(22) PCT Filed: Dec. 10, 2002
(86) PCT No.: PCT/EP02/14019

(30) Foreign Application Priority Data
Dec. 12, 2001 (DE) 10161143.9

(51) Int. Cl. A61K 48/00

(52) U.S. Cl. 514/44

(57) ABSTRACT

The invention relates to a therapeutic and prophylactic agent for malignant diseases, by phenotype suppression induced by ds-oligoribonucleotide and/or ds-mRNA or a ds-mRNA fragment, and to methods and the manufacture thereof, and for testing and monitoring the effect and for selecting and screening the relevant new drugs, whereby at least one of the phenotypically manifested properties is suppressed and the malignant cells or malignant tumors are not necessarily killed or eliminated but are returned to a benign state. The ds-oligoribonucleotide and/or the ds-mRNA or the ds-mRNA fragment, which are complimentary to one specific sequence of the gene which is activated in the cell on the completion of the malignant transformation and which is responsible for the selection and for the intracellular and transmembrane movement of the transcripts of at least one gene of those genes that are active or activated in the malignant cells, are manufactured for use, at least periodically, as a universal therapeutic agent or prophylactic agent in patients with malignant cells or malignant tumors, or in persons at increased risk of developing cancer, alone or at least with one of the ds-oligoribonucleotides, the ds-mRNA or the ds-mRNA fragments which are complementary to one of the gene-specific sequences of at least one gene of those genes which contribute directly or indirectly to the malignant process, irrespective of whether these genes also occur in normal cells, or are responsible for the increased risk of cancer, are in symmetry synthesized in vitro at a limited length.

The invention relates to a therapeutic and prophylactic agent for malignant diseases by phenotype suppression induced by ds-oligoribonucleotide and/or ds-mRNA or a ds-mRNA fragment, and methods for the manufacture thereof and for testing and monitoring the effect thereof and for selecting, screening and testing new drugs, whereby one of the already phenotypically manifested properties is suppressed and the malign cells or the malignant tumors are not necessarily killed or eliminated but are returned to a benign state, whereby the ds-oligoribonucleotide and/or ds-mRNA or the ds-mRNA fragment, which are complimentary to one of the specific sequences of the gene which becomes active in the cell on the completion of the malignant transformation and which is responsible through its peptide product for the selection and for the intracellular and transmembrane movement of at least a substantial amount of the produced transcripts of at least one gene of those genes that are active or activated in the malignant cells, are manufactured for use, at least periodically, as a universal therapeutic agent or prophylactic agent in patients with malignant cells or malignant tumors, or in persons at increased risk of developing cancer, or the ds-oligoribonucleotide or ds-mRNA or a ds-mRNA fragment, which are complimentary to a specific sequence or to the sequence, which is affected by at least one of the inherited, inborn or acquired genetic changes, of at least one gene of those genes which contribute directly or indirectly to the malignant process, regardless of whether these genes also occur in normal cells, or are responsible for the increased risk of cancer, are manufactured for use as a therapeutic or prophylactic agent in patients with malignant cells or with malignant tumors or in persons with a high risk of developing cancer, either alone or in combination with each other, preferably with the above-mentioned (1.a.) universal therapeutic or prophylactic agent and thus to enable the best individually-tailored, genetically-based therapy for the patient in question.

Despite the great progress of the last few years In the field of cancer therapy and prevention, cancer remains the third most frequent cause of death, causing patients great physical and mental suffering and placing a psychological and financial burden on the family. The annual cost of treating cancer runs into billions and the economic and social costs to the general public is substantially higher still. Cancer treatments are not only expensive but cause unpleasant stresses and complications. Suffering cancer remains a real tragedy. The situation of those people who are still healthy, but who are at high risk of developing cancer as a result of inherited, inborn or acquired genetic changes, is also sad because they are forced to live in a constant state of fear of developing a fast-developing cancer disease in most cases at a very young age without any possibility of guaranteed prevention. Bilateral mammectomies must always be carried out in young women in order to remove the danger of cancer, to avoid a cancerous disease and to free the patient from the psychologically unbearable state of anxiety. There is therefore sufficient cause to open new avenues in the field of cancer therapy and cancer prophylaxis. During the studies of the uptake of RNA and the fragments thereof by different cells, it was unexpectedly observed that ds-mRNA, in particular ds-oligoribonucleotide, causes In the cells and in the extracellular fluid, a drastic decrease of the peptide or protein product of the gone to which the oligoribonucleotide or ds-mRNA are complementary, while the concentration of other proteins remains unchanged and the relevant gene shows no changes. At a later stage, the malignant morphology of this cell was regressed gradually back to normal and the growth was slowed considerably without any genetic change.

This phenomenon corresponds to the phenotype suppression induced by the above mentioned substances. The cells with suppressed malignant phenotype have ended their lifespan as a result of the normal aging process, probably as a result of the normal controlled cell death mechanism, or continued their lifespan harmlessly as virtually normal cells, or the tumors continued to exist quasi as benign adenomas.

Similar phenomena were observed with the gene activated in the cell on completion of the malignant transformation and which is responsible through its peptide product for the selection and for the intracellular and transmembrane movement of at least a substantial amount of the produced transcripts of at least one gene of those genes which are active or activated in malignant cells. Since this phenomenon was induced in vitro, this indicates that the emergence of this phenomenon does not require the involvement of the host organism.

Therefore theme substances are used consequently as a therapeutic agent for treating malignant diseases or for prophylaxis in persons who are still healthy, but who have a high risk of developing cancer due to inherited or inborn or acquired genetic change(s). That is the task of this invention.

The invention has several advantages. The main advantage is that it presents a universal therapeutic or prophylactic agent. Another additional important advantage is that gene mutation or other genetic change of the gene concerned cannot induce therapy resistance because the transformed peptide product cannot fulfill its mRNA moving function in the malignant cell. The malignant cell would thus maneuver into the same situation, which is what is really intended with this therapeutic agent concerned. Preliminary studies indicate that the inhibition of this gene in the manner detailed above increased the mRNA content in the nucleus.

An important advantage of the invention is that it describes a universal method suitable for the manufacture of all ds-oligoribonucleotide therapeutic and prophylactic agents with optimum effectiveness in phenotype suppression that does not have to be re-invented for each therapeutic and prophylactic agent. The invention thus enables the production of many such therapeutic and prophylactic agents which can then be tailored, either together or alone, but preferably with the universal therapeutic or prophylactic agent detailed in claim 1 at a., to provide an individual ideal combination for each patient according to the individual genetic conditions of the malignant cells or malignant tumors. This is also an important advantage of the invention. The information necessary for this relating to the genetic basis of the malignant alteration in the cells or in the malignant tumors of each patient can be given by the in vitro test methods of the invention before, during and after the therapy.

A further important advantage of the invention is its proposal of such a therapeutic and prophylactic agent directed against such target genes which also occur in normal cells and function normally, because the products of these normal genes can be used during or after therapy according to the lowest physiological need, particularly if the product of these genes exerts its effect through autocrine, paracrine or endocrine mechanisms Secondly, a therapy-free period can be arranged to prevent or reduce the potential negative effect on the normal cells.

After an effective ds-oligoribonucleotide therapy, there follows a therapy-free period lasting for weeks, months or even years without a relapse or with halted progression, for example a therapy with ds-oligoribonucleotide of c.bcr, c.myc or of one of the genes of the Kras gene group, particularly long lasting after the combination of c.bcr and c.myc (one of lymphosarcoma, carcinoma of the stomach, pancreatic cancer, rectal cancer).

There are large individual variations with respect to the length of the therapy-tree period. The use of the therapeutic and prophylactic agent according to the invention allows the increased intensity of the various receptors to be reduced, thus preventing the abnormal stimulation of growth of the malignant cells or malignant tumors. The use of a purposefully arranged therapy-free period to reduce or prevent a potential negative effect on the normal cells is also a major advantage of the invention. The epidermal growth factor, erb gene product receptor, receptor HER or B cell factor receptors such as Hodgkin's lymphoma or Hodgkin's cell antigen (K1-a or similar relevant genes), TGF etc. should be mentioned in this regard.

A further advantage of the invention is that the therapeutic and prophylactic agent described in claim 1 at a. can inhibit or reduce, or even stop the development and progression of muscular atrophy in malignant diseases. The production of the peptide responsible for the movement of mRNA is prevented and no protein with mRNA is released from the malignant cells or reaches the extracellular body fluids or the circulation and cannot exert its dysfunctional effect or inhibit muscle cell development or prevent the production of muscle protein. The production of the peptide product of the gene described in claim 1, point a., is inhibited and thus the protein with mRNA cannot reach the extracellular body fluids or the circulation and thus cannot exert its disruptive effect on muscle function and delay or reduce the development of the muscle cells and/or the production of the muscle proteins.

The prevention of the function of the gene mentioned in claim 1 at a. by the therapeutic agent of the invention has an advantageous effect outside of the cancer cells, particularly on the defensive cells of the host organism. Due to the fact that mRNA with the peptide of this gene cannot leave the cancer cells, they cannot be taken up by the defensive cells of the host and therefore they need not produce that which is encoded in the taken up mRNA and is advantageous for the cancer cells, and they can fulfil their immunological function. That is also an advantage of the invention.

This phenomenon was observed by the applicant in the mid-sixties while studying the phagocytosis of the cold precipitates of the serum proteins in malignant diseases, its general validity for malignant diseases was recognized and examined and these experiences of manufacturing the relevant human vaccines considered when the development of molecular biology or molecular genetics enabled mass productions of human peptides and proteins.

Proteins, peptides or factors responsible for muscular atrophy are processed from the malignant cell extract or the extracellular body fluids or the cell-free culture media using the above-described method of the invention and the effect thereof on the development of the muscle cells and on the production of muscle proteins can be tested and is tested using the appropriate method of the invention, namely in myoblast cultures or in muscle cell cultures or muscle tissue cultures.

The object of the invention is a therapeutic and prophylactic agent for malignant diseases, by phenotype suppression Induced by ds-oligoribonucleotide and ds-mRNA or a ds-mRNA fragment, and methods for the manufacture thereof and for testing and monitoring the effect thereof and for selecting and screening new relevant drugs, whereby at least the phenotypically manifested properties are suppressed and whereby the malignant cells or malignant tumors are not necessarily killed or eliminated but are returned to a benign state, whereby the ds-oligoribonucleotide and/or ds-mRNA or the ds-mRNA fragment, which are complimentary to one of the specific sequences of the gene which is activated on the completion of the malignant alteration in the cell through its translational product, and is responsible for the selection and for the intracellular and transmembrane movement transcript of at least mRNA which are activated in the malignant cell, are manufactured and are used, at least periodically, as a universal therapeutic or prophylactic agent in patients with malignant cells or malignant tumors, or in persons at high risk of developing cancer, alone or in combination with at least one of the ds-oligoribonucleotides and/or one of the ds-mRNA or one of the ds-mRNA fragments, which are complimentary to one of the specific sequences or to the sequence affected by at least one of the inherited, inborn or acquired genetic changes, of at least one gene of those genes that contribute directly or indirectly to the malignant process irrespective of whether these genes also occur in normal cells or are responsible for the high risk of developing cancer, are manufactured to be used at least periodically in patients as a therapeutic and prophylactic agent combined with each other but preferably with the therapeutic and prophylactic agent detailed above at 1a. The best individual, gene-based therapeutic agent combination is thus ideally tailored for the patent in question. With the method according to the invention, these therapeutic agents are already examined and tested before and during therapy for their effect in vitro in malignant cell cultures or with malignant cells, and if available, with the tumor tissue of the patient in question, in order to thereby avoid needless administration of ineffective drugs and to protect patients from unnecessary harm, whereby the malignant cells or malignant tumors are not necessarily killed or eliminated but are returned to a benign state and the tumors can continue to exist virtually as benign adenomas without there being any damaging effect on the host organism.

This problem is solved by selection, isolation and manufacture of the mRNA of those genes which are still unknown and/or by molecular cloning and/or by in vitro enzymatic multiplication, which is converted into a double helix by annealing and used in this form as a therapeutic or prophylactic agent or by fragments produced by protective controlled fragmentation, whereby fragments separated by acrylamide gel electrophoresis are tested in vitro for their effectiveness, and the effective fragment is biologically or enzymatically multiplied and either is used in double helix form as a therapeutic agent or the genetic sequence of the effective fragment or the entire mRNA itself is determined using automated sequencers and one of the specific sequences as in the genes with the known genetic code corresponding to the genetic code, symmetrical to the mutated codon or the point of contact between two genes, which are fused, or to the codon in the middle of the sequence which is not affected by genetic change is synthesized at a limited length of at least 19 codons but not substantially more than 23 codons and preferably 21 codons, preferably in an automized synthesizer.

In order to select mRNA, the peptide product thereof is isolated using the method according to the invention, to use this to manufacture immune serum by immunizing the animals (rat, rabbit). The immune serum is bonded monospecifically to the peptide product of the mRNA using Sepharose immunoaffinity chromatography to the normal cell extract and the IgG fraction is separated using protein A Sepharose affinity chromatography and thus the microsome-mRNA complex and the peptide product freshly formed thereon is precipitated from the malignant cell lysate, and the mRNA is separated or isolated and multiplied biologically or by in vitro enzymatic multiplication and is used in double helix form either for therapy or for further manufacturing processes, in this case to test the effectiveness of fragments separated after controlled protective enzymatic splitting using acrylamide gel electrophoresis, using the method of the invention. The effective fragment undergoes in vitro enzymatic multiplication and is used in double helix form for therapy or prophylaxis, or its genetic sequence or the genetic sequence of the mRNA reaction (Amersham, Sigma). The mRNA and the DNA dissolved In BPS are brought into ss form by heat shock at 95° C., quickly cooled, then incubated at 50° C., so that RNA and the DNA form hybrid complexes with each other. These hybrid complexes are treated using restriction enzymes, the released parts of the RNA are separated using RNase and the remaining RNA fragments are separated in acrylamide (2%) gel electrophoresis. After the fragments undergo in vitro enzymatic multiplication, they are subjected to in vitro examination of their effectiveness. The effective fragment is multiplied in vitro by enzymatic multiplication and the product is manufactured biologically. The product of the in vitro enzymatic multiplication or molecular cloning is converted into a double helix by annealing for use in therapy or prophylaxis treatment or the product is subjected to further examinations or manufacturing processes.

The genetic sequence of the effective fragment or of the mRNA molecule is preferably determined by an automated sequencer or, if the peptide product has been isolated, by the amino acid sequencing thereof and, as in the case of genes with known genetic sequences, is preferably chemically synthesized in an automated synthesizer, according to the appropriate code with respect to the number of codons in symmetry to the mutated codon, or to the point of contact between two genes, which are fused, or to the codon in the middle of the gene-specific sequence which is not affected by genetic change, at a limited length of at least 19 codons but not substantially more than 23 codons, preferably 21 codons. The product is converted into double helix form by annealing for use either as a therapeutic or prophylactic agent.

The above-mentioned method for the selection of the mRNA described in the invention can be used for all other mRNA selections, and for this purpose the protein or peptide product of the mRNA must firstly be isolated, in order to thereby produce monospecific antibodies. The cell lysate of the malignant cells is firstly subjected to immunoaffinity chromatography with the lysate of normal cells, and is carried out according to the manufacturer's instructions. The absorbency of the fractions is measured at 260 and 280 mu and registered as such. The fractions with the increased 260/280 ratio are collected separately and chemically examined and the RNA is isolated from the peptide and the lipids are removed.

The first and second larger dissolved fractions contain a peptide with a molecular weight of 1200 and a 25 S poly-A positive RNA which stimulates and/or controls, in a cell-free system for protein synthesis, the incorporation of radioactively labeled amino acids in the protein or peptide as mRNA. The first large fraction still contains lipids.

The peptide is firstly released from the lipids and the RNA by chloroform/methanol extraction, the isolated fractions are suspended for this purpose in distilled water and are mixed with 5 ml chloroform/methanol 2:1 vol/vol and extracted at 22° C. with constant vortexing for 3 hours and then centrifuged at 4° C. at 10000×g for 10 minutes. The residue is re-extracted using 2.5 ml chloroform/methanol 1:1.5 vol/vol. The organically soluble fractions contain lipid and peptide and were free of RNA, they are collected and further extracted. The organically insoluble fraction is rich in RNA and is separately subjected to further separation.

The organically soluble phases are dissolved in 2 ml chloroform/methanol 2:1 vol/vol and have 2 ml diethylether added thereto and are incubated at 4° C. for 10 hours and the peptide-rich precipitate is separated by centrifugation at 10000×g for 10 minutes and further extracted using 2 ml HCl (pH 2.0) acidified chloroform/methanol 1:1 vol/vol and the extraction is repeated twice. The delipidized precipitate is dissolved in distilled water and is mixed with Freund's adjuvant for immunization. Rats or rabbits are immunized i.m. at least twice with a space of 4-8 weeks between immunizations.

The IgG fraction is chromatographically isolated from the rat immune serum with protein A Sepharose and the IgG fraction is subjected to immunoaffinity chromatography, whereby the IgG fraction of the immunoserums is activated using Cn.Br., whereby the cell extract of the normal cells is bonded to the Cn.Br.-activated Sepharose and the IgG fraction of the immune serums is specifically adsorbed with this Sepharose either by batch technology or by column chromatography and the absorbed fraction is eluted to obtain a monospecific IgG antibody traction. This monospecific IgG antibody fraction is bonded to Cn.Br.-activated Sepharose 6 B in order to isolate the peptide according to its antigen specificity in each fraction or cell extracts or extracellular fluids and cell-free culture media using immunoabsorption chromatography in accordance with the instructions of the manufacturer (Pharmacia). The Bio-Gel product B1 and B10 mesh 100400 should be used according to the instructions of the manufacturer (Bio Rad) to separate and characterize small peptides.

Isolation of polyarpositive A mRNA is carried out using poly-U Sepharose (Amersham) or similar methods (Sigma).

The method described above is not only suitable for the isolation of the peptide mentioned in claim 1 at a., but also for other peptides such as the protein or peptide detailed in claim 7 at b. or other protein or peptide products, if a test method can demonstrate the specific effect of these protein or peptide products. The invention proposes such a test method, whereby the prevention of the growth of the myoblast cell culture or the effect on the incorporation of radioactively labelled amino acids in the muscle proteins, preferably in myosin, is to be tested; in this case the radioactivity in cell extracts of muscle tissue cultures with the cultures treated with test protein or peptide compared with untreated controls which can be measured for the amount of radioactivity.

The test method of the invention has made possible the detection of factors that play a role in the muscular atrophy mechanism in malignant diseases and therefore allows the spotlight to deservedly fall upon this aspect of malignant diseases which has until now been almost totally neglected, that muscular atrophy does not only affect the muscles of the musculoskeletal system but also the respiratory system and the cardiac muscle and leads to death in a substantial percentage of cases.

The mechanism mentioned in claim 7 at a. with the peptide and RNA particularly causes malfunctions in other tissues and other organs and substantially contributes to the so-called fatigue syndrome in malignant diseases, even long before the malignant disease can be detected either clinically or using other test methods. The therapeutic and prophylactic agent in claim 1 at a. also helps to prevent or delay this successive phenomenon which progresses constantly during the illness.

The therapeutic and prophylactic agents according to the invention enable a genetically-based, individual, ideal combination which can be tailored individually for the patient in question, even before the beginning of therapy, since the invention proposes the in vitro effectiveness test method necessary for this. This method can be used to examine not only the established malignant cell lines for the effectiveness of the relevant therapeutic and prophylactic agents, but also the malignant cells or, if obtainable, the tumor tissue of the patient in question. The effectiveness of the intended therapeutic agent or therapeutic agent combination on the individual's tumor tissue or the malignant cells can be examined directly before the beginning of therapy, and superfluous therapy attempts using unsuitable therapeutic agents or therapeutic agent combinations, and therefore unnecessary harm to the patient and unnecessary costs, can thus be avoided.

This test method can be combined with the rolling drum semi-micro cell/tissue culture technique and a large number of specimens can thus be tested quickly and cost-effectively or hundreds and thousands of substances can also be examined quickly and cost-effectively in series for their effectiveness as anti-malignancy drugs, selected and screened. The rolling drum semi-micro cell/tissue culture technique was published decades ago in English-language specialist literature by the applicant and a partner. At that point this technique was originally conceived and used to examine viruses, but the applicant also tried it with human malignant cells and malignant tumor tissue and found it to be suitable, but at that time there was no suitable test method which could specifically and reliably demonstrate the effect of the intended drugs. The invention now proposes this test method. The rolling drum semi-micro cell/tissue culture technique, which was originally conceived for examining viruses, can thus now be used for examining clinical material of new pharmaceutical substances.

Several years after publication of the rolling drum semi-micro cell tissue culture method, the apparatus and technique were marketed by an overseas firm and recommended with a photo of the device in one of the best scientific journals in the world as a scientific instrument for examining viruses.

By detecting the gene product detailed in claim 1, at point a., and its mRNA, it is possible to measure the effect of almost all effective anti-cancer cell or anti-malignant tumor therapeutic agents, long before morphological changes occur in vitro; the RNA release in the cell-free culture media reacts particularly quickly, even within hours, and this can be detected or measured very sensitively by in vitro enzymatic multiplication by means of the RNA polymerase chain reaction or after reverse transcription by means of DNA polymerase chain reaction. In this case, where RNA release is measured, universal primers should be used. This is a general test method for almost all effective anti-cancer drugs in vitro. However, the invention proposes a specific effect detection method. In this case, the RNA release of said gene is directed against the oligoribonucleotide therapeutic agent according to the invention. This is more specific and even more sensitive than the above effectiveness test method. In this case, specific primers should be used according to the genetic sequence of the gene targeted by the ds-oligoribonucleotide therapeutic agent according to the invention.

The protein or peptide product of the gene targeted by the therapeutic agent is suitable for the above purposes, such as RNA determination, but the reaction to the effect of the therapeutic agent becomes slower. The protein or peptide product can be verified immunocytochemically in the cells and in the tissue cultures using immunofluorescence methods, except for the cell extract or tissue extract of cultures and cell-free culture media and in extracellular body fluids, the protein or peptide product can preferably be detected immunologically preferably using the following methods: The peptides or proteins in the extractum or in the cell-free culture fluid or in one of the extracellular body fluids of living beings are bonded on a water-insoluble substrate Affigel 702 Beads (Bio Rad), washed and used according to the manufacturers instructions. 0.2 ml with 0.2 ml of anti-target gene peptide, such as amyC, or anti-target protein products, such as amyC of mouse or rabbit immune serum is then admixed and incubated at 37° C. for 4 hours or at 4° C. for 18 hours, washed and incubated with anti-mouse or anti-rabbit immunoglobulin, preferably immune serum, but with the immunoglobulin fraction thereof, which is conjugated with an enzyme (peroxidase, alkaline phosphatase), washed, then the enzyme substrate is admixed and evaluated using color reaction after incubation. Exact quantitative determination can be carried out in a similar manner using serial dilutions of the specimens. The substances are tested for side effects in cell cultures and/or living organisms before they are used in the patient. Genetically modified animals such as the Oncomouse, which are also commercially available (Harvard, Boston and Du Pont, Del., U.S.A.), or animals with impaired immune systems (mouse, rat) are particularly suitable for this purpose. The therapeutic and prophylactic agent can be administered to the patient parenterally, per os and locally or in a combination of the three application methods. With intravenous administration, the dose is 0.3-8.0 mg per kg of body weight every 4-7 days. The therapy-free periods can be trialed to initiate after just 3, 4 or 6 weeks of the therapy. If well tolerated, the dose can be increased gradually. Fever can occur with I.v. administration of greater than 5 mg/kg, but this can be kept under control using suitable anti-fever therapy or pre-therapy.

The invention uses for the first time human fibrin sponge as a carrier, reservoir and protective substrate for the anti-malignancy therapeutic agents, such as the therapeutic agents of the invention, for local treatment in order to substantially strengthen the effect of the systemic treatment locally (human fibrin sponge therapy). In the case of cancer of the esophagus and carcinoma of the stomach, small pieces of fibrin sponges, which are soaked with the therapeutic agent of the invention which is mixed with nuclease inhibitors, are swallowed several times a day (oral fibrin sponge therapy). In the case of local treatment, human fibrin sponge pieces, in the shape and length suitable for the intended purpose, are also soaked with the therapeutic agent of the invention in combination with nuclease inhibitors and are placed non-invasively in the body cavities (such as the oral cavity) or in the organs covered with mucous membranes (rectum, sigmoid, vagina, cervix, uterus) directly on the malignant alterations to substantially increase the local effect of the systemic treatment or to carry out this local treatment alone in the systemic therapy-free period. The use of human fibrin sponge therapy also significantly widens the range of possible local uses of other anti-cancer drugs.

The therapeutic agent can be administered directly into the pancreas, via a tube inserted endoscopically into the pancreatic duct, particularly at the start of the systemic therapy as an introductory therapy. If the tumor or larger metastases are localized, the therapeutic agent can be injected directly into these tumors or metastases under ultrasound tomography control, even as an initial therapy. Aerosol spray inhalation therapy for lung cancer can also be considered, alone or in the form of liposomes. Percutaneous application using lipids or liposomes is also possible. The genes are changed by mutation in a substantial percentage of malignant diseases, which plays a fundamental role in the development and continuation of the malignant process, the genes of the ras gene family being particularly frequently affected through mutation in position 12, 13 and 61. In this case, oligoribonucleotides are synthesized with respect to the number of the codons in symmetry to the mutated codon, e.g. in the case of mutation at codon 12, 10 codons are synthesized in the order of the genetic sequence and then the muted codon and thereafter 10 codons are synthesized In the order of the genetic sequence. In the case of genes, which are fused, of the synthesized de-oligoribonucleotide, synthesizing occurs with respect to the number of codons, symmetrically to the point of contact of the two genes, which are fused, as for the fusion of the bcr and abl genes in myeloid leukemia. This fusion relates to the bcr and abl genes in chronic myeloid leukemia. In the course of the illness, further genetic changes, mainly gene mutations, also arise, preferably in genes of the ras gene family and together trigger the serious or even fatal complication of the acute plastid transformation crisis. The ds-oligoribonucleotides are synthesized with respect to this additional gene change or mutation of the invention detailed above and are used as a therapeutic agent and also for the treatment of this mostly fatal complication before the crisis stage or for the prevention thereof.

Point mutations often occur in the most frequent types of cancer such as carcinoma of the stomach, pancreatic cancer, colon cancer, rectal cancer, bronchial cancer and breast cancer. Using the method of the invention, ds-oligoribonucleotides are manufactured for these gene mutations and are successfully used for therapy or prophylaxis treatment for these or other malignant diseases.

If, due to mutation, resistance is developed during therapy to the therapeutic agent used, a ds-oligoribonucleotide, which is complementary to the sequence of the sequence affected by the new mutation, can be manufactured using the synthesizing method of the invention and used as a therapeutic agent, to thereby overcome the therapy resistance which has arisen. This possibility can be useful, whereby, due to mutation of the target gene, resistance occurs following a good therapeutic effect. The ds-oligoribonucleotide which is complementary to one of the gene sequences of the Hodgkin antigen family (K1a) is synthesized according to the synthesizing method of the invention for therapy purposes and/or to prevent relapse.

The foreign genes, which were artificially introduced and integrated into the gene units by the gene unit during a viral infection or artificially due to therapy attempts, play a double role:
1. They will cause gene changes by means of the uptake of these foreign genes in their own genetic structure of the cells and the cell will thus have a high risk of malignant alteration. These genetic changes are mainly caused by mutation.
2. On the other hand, a chronic infection caused by this settling in of foreign genes often leads to a general risk of developing cancer.

The ds-oligoribonucleotides, which are complementary to these virus genes, or ds-oligoribonucleotides, which are complementary to the sequences changed by this gene settlement, can be synthesized for therapy purposes or for prophylaxis treatment. Viruses such as papillomaviruses (HPV 16, 18), Epstein-Barr virus, HIV, Hepatitis C viruses and Murine Mammary Tumor virus (MMTV) as well as some types of adenoviruses should be mentioned in this respect and ds-oligoribonucleotides complementary to the gene-specific sequence are synthesized and used for therapy and prophylaxis treatment.

The telomerase enzyme plays an Important part In the immortalization of malign cells; de-oligoribonucleotide, which is complementary to the genetic sequence thereof, can also be synthesized according to the methods of the invention in order to be used with others for therapy or prophylactic purposes.

Fertility problems can occur in patients, which are treated with the oligoribonucleotide that belongs to the gene-specific sequence of the telomerase enzyme. These can be prevented by banking sperm or egg cells before therapy.

In combination with various complementary di-oligoribonucleotides as therapeutic or prophylactic agents of the invention, the most optimal genetically-based therapy can be Individually arranged for each patient. The malignant cells or tumors are thus not necessarily killed or eliminated, but are returned to a benign state and the malignant tumors can continue to exist almost as benign adenomas, without exerting any damaging effect on the host organism.

A fatal illness is thus transformed into a chronic condition, which enables a good quality of life, without any shortening of life expectancy.

That is the aim of this invention.

1. A therapeutic and prophylactic agent for malignant diseases, by phenotype suppression induced by ds-oligoribonucleotide and/or ds-mRNA or a ds-mRNA fragment, and methods for the manufacture thereof and for testing and monitoring the effect thereof and for screening and selecting new drugs, whereby at least one of the already phenotypically manifested properties is suppressed and the malignant cells or malignant tumors are not necessarily killed or eliminated but are returned to a benign state, whereby
a. the ds-oligoribonucleotide and/or ds-mRNA or the ds-mRNA fragment, which are complimentary to one of the specific sequences of the gene which becomes active in the cell on the completion of the malignant transformation which is responsible through its peptide products for the selection and for the intracellular and transmembrane movement of at least a substantial amount of the produced transcripts of at least one gene of those genes that are active or activated in the malignant cell, are manufactured for use, at least periodically, as a universal therapeutic agent or prophylactic agent in patients with malignant cells or malignant tumors, or in persons at increased risk of developing cancer, and/or
b. the ds-oligoribonucleotide and/or ds-mRNA or ds-mRNA fragment, which are complimentary to one of the specific sequences or to the sequence, which is affected by at least one of the inherited, inborn or acquired genetic changes, of at least one gene of those genes which contribute directly or indirectly to the malignant process or are responsible for the increased risk of cancer, are manufactured for use, at least periodically, as a therapeutic agent in patients with malignant cells or with malignant tumors or for prophylaxis in persons with a high risk of developing cancer, either alone or in combination with each other (at least 2 of them), preferably with the above-mentioned universal therapeutic or prophylactic agent,
c. the RNA, if it is not yet known, is selected from the cell lysate of the malignant cells and is either manufactured biologically by means of molecular cloning or in vitro by enzymatic multiplication, and is then subjected to a controlled protective enzymatic fragmentation, the fragments being separated and being subjected to in vitro study of their effect, the effective fragment is multiplied biologically or in vitro and the sequence thereof is determined and the sequence or sequences thereof and the specific sequence, as in the genes with a known genetic code, is synthesized biologically or preferably chemically in vitro at a limited length and is then converted into a double helix by annealing.

2. The therapeutic and prophylactic agent according to claim 1, characterized in that the RNA, if it is still not known, is selected from the cell lysate of the malignant cells and is multiplied by means of molecular cloning or in vitro by enzymatic multiplication, and is either used in double helix form for therapy or for prophylaxis or is subjected to a controlled protective enzymatic fragmentation, the fragments are subjected to in vitro study of their effect following separation, the effective fragment is subjected to biological or in vitro enzymatic multiplication and is used in double helix form as a therapeutic or prophylactic agent or the genetic sequence of the fragment or of the RNA itself or the amino acid sequence of the peptide products thereof is determined and, as in the case of genes with known genetic code, preferably chemically synthesized at a limited length in an automated synthesizer with respect to the number of codons in symmetry to the mutated codon, or to the point of contact between two genes, which are fused, or to the codon in the middle, of the gene sequence which is not affected by genetic change, and is converted into double helix form by annealing,
a. and is administered systemically or locally for therapy or for prophylaxis,
b. fibrin sponge, preferably human fibrin sponge, is used as a vehicle for the therapeutic agent and as a reservoir, whereby pieces of the fibrin sponge are cut into an appropriate shape and length, then pushed in the solution of the therapeutic agent, mixed with nuclease inhibitors, in order for the sponge to be soaked in this therapeutic solution, and placed directly at malignant alterations either in body cavities or in organs covered with mucous membranes or, in the case of cancer of the esophagus and carcinoma of the stomach, is used several times daily as a local treatment by swallowing small pieces of the abovementioned prepared sponge, in order to locally strengthen the effect of the systemically administered therapeutic agent according to the invention: fibrin sponge therapy, oral fibrin sponge therapy
c. in the case of pancreatic cancer, the therapeutic agent of the invention, preferably as liposome, is administered directly into the pancreas through a tube, inserted endoscopically into the pancreatic duct.

3. The therapeutic and/or prophylactic agent according to claim 1, 2, characterized in that the ds-oligoribonucleotide or ds-mRNA or ds-mRNA fragment is complementary:
a. to the RNA or to one of the fragments thereof, said at a. in claim 1, which is responsible through its peptide product for selection and for intracellular and transmembrane movement of the transcript of at least one gene of those genes which are activated in the malignant cells, or
b. to one of the specific sequences or to the sequence, affected by at least one of the genetic changes, of at least one gene of those genes which are directly or indirectly implicated in the malignant cells in the controlled cell death mechanism or directly or indirectly contribute to the immortality of the malign cells,
c. or to one of the gene-specific sequences of at least one gene of those genes which are directly or indirectly implicated in the production of the therapy resistance factor.

4. The therapeutic and prophylactic agent according to claim 1, 2, 3, characterized in that ds-oligoribonucleotide or ds-mRNA or ds-mRNA-fragment is complementary to the gene sequence, which is affected by mutation, depletion or gene fusion, irrespective of whether the gene products have normal functions, abnormal functions or no functions at all.

5. The therapeutic and prophylactic agent according to claim 1, 2, 3, 4, characterized in that the ds-oligoribonucleotide and/or the ds-mRNA or the ds-mRNA fragment is complementary to a specific sequence of at least one gene of those genes which are not themselves affected by genetic changes but which malfunction due to genetic dysregulation or by amplification, these genes also occuring in the normal functioning condition in normal cells and the product of these normal functioning genes, particularly when the products thereof exert their effect through autocrine, paracrine or endocrine mechanisms, is used at least periodically at the same time as the therapeutic or prophylactic agent or in succession in the therapy-free period according to the lowest physiological requirement.

6. The therapeutic and prophylactic agent according to claim 1, 2, 3, 4, 5, characterized in that the ds-oligoribonucleotide and/or the ds-mRNA or the ds-mRNA fragment is complementary to one of the specific sequences of those genes which produce qualitatively or quantitatively malfunctioning receptors, such as the epidermal growth factor receptor, with erb-B, receptor R or B cell factor receptors such as Hodgkin's antigen or Hodgkin's cell antigen (K1a) or TGS receptor etc. or to the specific sequence of the genes which produce these growth factors.

7. The therapeutic and prophylactic agent according to claim 1, 2, 3, 4, 5, 6, characterized in that ds-oligoribonucleotide and/or the ds-mRNA or the ds-mRNA fragment is complementary to one of the specific sequences of those genes which is responsible for muscular atrophy which occurs in malignant diseases:
a. the gene said in part a. of claim 1, whose peptide product with mRNA reaches the extracellular body fluids and the circulation, exerts a disruptive effect on muscle function, hinders the development of muscle cells and delays or prevents the production of muscle proteins, furthermore causes disorders, and particularly dysfunction, in other tissues or other organs, and considerably contributes, together with muscle dysfunction, to the so-called fatigue syndrome long before the clinical manifestation of the malignant disease or its diagnosis,
b. the gene which, in protein or peptide product present in the patient's extracellular body fluids, exerts a similar effect on muscle development and function and contributes to the muscular atrophy that occurs with malignant diseases,
c. in vitro examination of this effect enables the method of the invention to detect the effect of the factors mentioned above at a. and b. on the myoblast cell cultures or on the incorporation of radioactive labelled amino acids in the muscle proteins, preferably in myosin, is reduced.

8. The therapeutic and prophylactic agent according to claim 1, 2, 3, 4, 5, 6, 7, characterized in that the therapeutic or prophylactic agent given at a. in claim 1 prevents the production of the peptide, which moves mRNA intracellular and transmembrane in the malignant cells. The mRNA cannot leave the malignant cells, either protected or unprotected, and thus the defensive cells of the host cannot take up the mRNA and therefore need not produce that which is encoded in the mRNA and is advantageous for the malignant cells and furthermore released and can thus better fulfil their immunological function.

9. The therapeutic and prophylactic agent according to claim 1, 2, 3, 4, 5, 6, 7, 8, characterized in that the ds-oligoribonucleotide and/or the ds-mRNA or the ds-mRNA fragment is complementary to a genetic sequence of at least one gene of those genes that are affected by genetic changes and/or malfunctions due to the settling in or integration of the genetic material introduced naturally, such as viral infections, or artificially for therapy purposes, or to the settled genetic material such as human papillomavirus (HPV 16, 18), Hepatitis C virus, HIV, Epstein-Barr virus and some types of adenoviruses etc. and to the artificially introduced and integrated genetic materials which were introduced into the genome of the cell for therapeutic purposes and have then induced a malignant disease in these cells by means of malignant alteration.

10. The therapeutic and prophylactic agent according to claim 1, 2, 3, 4, 5, 6, 7, 8, 9, characterized in that the therapeutic and prophylactic agent are examined for their effect in normal and malignant cell cultures or tissue cultures, whereby
a. the peptide or protein product of the suppressed gene function is preferably examined immunologically using the immunofluorescence method or is quantatively determined in the cell or cell cultures,
b. and/or is preferably examined immunologically, using an Enzyme-Linked immunosorbent Assay (ELISA), or is quantitatively determined in the cell or tissue extract and/or in a cell-free culture medium and/or in extracellular body fluids such as blood plasma or in body fluids such as urine which can be obtained non-invasively, and/or
c. the mRNA in the above detailed (a. b.) specimens is, using in vitro enzymatic multiplication, identified or quantitatively determined, and/or
d. the malignant cell culture or malignant tissue cultures and, if obtainable, the malignant tumor tissue culture or tissue culture of the respective patients are examined or quantitatively determined, with the method given at a., b., c., before, during and after the therapy,
e. and used with the method given at a., b., c., d. for selecting, screening and testing new therapeutic and prophylactic agents. Particularly with the rolling drum semi micro cell and tissue culture methods can a large number of specimens be processed in series in a time and cost-effective manner, particularly combined with robotic machinery.
